(19) United States
(12) Patent Application Publication
(10) Pub. No.: US 2023/0086663 A1
(43) Pub. Date: Mar. 23, 2023

(54) ANTIMICROBIAL MATERIAL

(71) Applicant: Paravir Limited, Sudbury (GB)

(72) Inventors: Andrew Patchett, Basildon (GB); Kim Patchett, Grays (GB)

(21) Appl. No.: 17/989,346

(22) Filed: Nov. 17, 2022

Related U.S. Application Data
(63) Continuation of application No. PCT/GB2021/051203, filed on May 19, 2021.

(30) Foreign Application Priority Data
May 19, 2020 (GB) 2007392.0

(51) Int. Cl. A01N 59/08; A01N 25/12; A01N 25/10; A01N 59/00; A01P 1/00

(52) U.S. Cl. CPC A01N 59/08; A01N 25/12; A01N 25/10; A01N 59/00; A01P 1/00

(57) ABSTRACT

Disclosed is an antimicrobial air permeable substrate comprising a high concentration of a dry powder. Also disclosed is the use of the antimicrobial material to kill, denature or otherwise deactivate microbes, particularly airborne or droplet-borne microbes.

The invention also relates to a functionalised fabric that will deactivate air borne virus upon contact. In particular, it relates to a fabric within which is contained an active compound or compounds that have been demonstrated to deactivate air borne virus and other pathogens when said virus or pathogens make contact with the active compound within the fabric. The active compound or compounds described are harmless to humans, animals, marine and plant life and are prolifically available from sustainable resources.

### CROSS-REFERENCE TO RELATED APPLICATIONS

Continuation of International Application No. PCT/GB2021/051203 filed in May 19, 2021. Priority is claimed from British Patent Application No. 2007392.0 filed on May 19, 2020. Both the foregoing applications are incorporated herein by reference in their entirety.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### BACKGROUND

### Field of the Invention

The present invention relates to an antimicrobial air permeable substrate comprising a high concentration of a dry powder. The invention further relates to use of the antimicrobial material to kill, denature or otherwise deactivate microbes, particularly airborne or droplet-borne microbes.

The present invention relates to a functionalised fabric that will deactivate air borne virus upon contact. In particular, it relates to a fabric within which is contained an active compound or compounds that have been demonstrated to deactivate air borne virus and other pathogens when said virus or pathogens make contact with the active compound within the fabric. The active compound or compounds described are harmless to humans, animals, marine and plant life and are prolifically available from sustainable resources.

### Background to the Invention

Antimicrobial materials take many forms, from fabrics soaked in antimicrobial solutions to solid materials such as plastics impregnated with, or coated with, antimicrobial additives such as Microban®.

One area that it has become apparent that there is a lack of effective antimicrobial material is the healthcare sector, in particular, for use in personal protective equipment (PPE) and other materials or fabrics used in healthcare settings (such as privacy curtains).

Although, for example, there are many types of face masks currently commercially available to healthcare workers, care sector workers and the general public, it is thought that none of the current product offerings are able to deactivate viral or other pathogenic infections upon contact.

The coronavirus pandemic has led to the widespread use of face coverings in the general population and the lack of effective material for use in this area has become of considerable concern. Many types of masks claim to be antimicrobial, often by the inclusion of copper adhered to the fabric. However, whilst copper is known to have antimicrobial properties, there is no standard for ensuring that the amount of copper present has any effectiveness. Furthermore, it would be prohibitively expensive to include the amount of copper needed to reach a high level of effectiveness.

Alternatives to copper include use of filters of varying types such as carbon or HEPA (high efficiency particulate air) filters or use of other additives to the material such as zinc, silver and organic salts. Even then, many of the materials are proven to be antibacterial rather than antimicrobial and specifically not antiviral.

Use of simple salts such as sodium chloride has been suggested by Choi in WO2018/033793, however it has proven to be difficult to produce a material with a high enough concentration of salt (or any other active ingredient) to effectively deactivate microorganisms and viruses.

This is because prior art methods employ wetting techniques. Given that the saturation point of NaCl in water is 357 g per litre at 25° C. (which equates to 26.3% w/w) it is not surprising that this is the case. Furthermore, a lot of non-woven materials are inherently hydrophobic and would require a surfactant to help the saturated saline to penetrate the material. In practice, what happens is that, as the water evaporates, the salt crystalises on the surface of the material, rather than embedded within the material. This means it is easily lost from the material. Furthermore, since it is not possible to rewet the material without redissolving the salt, there are no means of increasing the salt concentration. Thus, wetting techniques are not practical for impregnating particles into substrates, particularly non-woven materials.

Thus, there is a need for effective antimicrobial materials with a high concentration of active ingredient, that do not contain potentially toxic materials, that uses readily available ingredients and has good efficacy against viruses as well as larger microbes (e.g. bacterial and fungi). It is under these conditions that the present invention has been devised.

The object of the present disclosure is to create a fabric that may be, among other things, incorporated into personal protective equipment (PPE) and in particular, face masks, so that the PPE not only acts as a filtering barrier to viral infection but deactivates said viral species upon contact thereby reducing the spread of infection of the virus.

### SUMMARY

According to a first aspect there is provided an antimicrobial air-permeable substrate in the range 5 gsm to 500 gsm comprising a dry powder with a maximum particle size of 500 μm in the amount of at least 20% w/w.

In a second aspect there is provided a multi-layered material comprising at least one layer of the substrate according to the present disclosure.

### BRIEF DESCRIPTION OF THE DRAWINGS

For a better understanding of the invention and to show how the same may be carried into effect, there will now be described by way of example only, specific embodiments, methods and processes according to the present invention with reference to the accompanying drawings in which:

FIG. 1 shows a schematic of a cross section through an example air-permeable antimicrobial substrate 20 according to the invention in which dry powder is indicated by 40 and fibres are indicated by 30.

FIG. 2 shows a mechanism for deactivating a microorganism, for example, a virus.

A shows a representation of a virus 1 in a droplet or aerosol 2.

B shows the virus 1 in the droplet or aerosol contacting dry powder 3 as comprised in the substrate of the invention and the dry powder solvating into the droplet to form a solution 4.

C shows the virus 1 in the solution droplet 5 and the increasing osmotic pressure 11 on the virus.

D shows increased concentration of the solution droplet 6 and further increase in osmotic pressure 11.

E shows further increased concentration of the solution droplet 7 which is now evaporating and yet further increase in osmotic pressure 11.

F shows hyperosmotic pressure as the droplet 8 evaporates further. Recrystallisation of the dry powder 12 causes lysis of the virus 10

FIG. 3 shows an example compartmentalisation pattern on the substrate of the invention.

FIG. 4 shows Results as Log₁₀PFU Sample⁻¹ against phi6 (enveloped bacteriophage)

### DETAILED DESCRIPTION

As employed herein, antimicrobial means an agent that kills microorganisms or stops their growth. In this context microorganism is intended to be interpreted broadly to encompass bacteria, archaea, fungi, protozoa and viruses, including pathogens. Antimicrobial agents can be grouped according to the microorganisms they act primarily against. For example, antibacterial, antiviral, antifungal. They can also be classified according to their function. Agents that kill microbes are microbicides (e.g. bactericidal), while those that merely inhibit their growth are called-static agents (e.g. bacteriostatic).

In one embodiment the antimicrobial is antiviral.

As employed herein, air-permeable substrate means any substrate that is air permeable. Examples of suitable substrates include, but are not limited to, fibrous and non-fibrous substrates, fabrics, including non-woven fabrics, open cell foam, composite materials, sintered composites and polypropylene (PP) printed scaffold.

In one embodiment the air-permeable substrate is a sheet material.

In one embodiment the sheet material is a fibrous material, such as a fabric.

Typically, the substrate is a material such as a non-woven material.

Non-woven as employed herein refers to a fabric-like material made from a staple fibre and long fibres bonded together by chemical, mechanical, heat or solvent treatment. The term is used in the textile manufacturing industry to denote fabrics, such as felt, which are neither woven nor knitted. Non-woven fabrics are broadly defined as sheet or web structures bonded together by entangling fibres or filaments (and by perforating films) mechanically, thermally or chemically. They are flat or tufted porous sheets that are made directly from separate fibres, molten plastic or plastic film. They are not made by weaving or knitting and do not require converting the fibres to yarn.

Non-woven materials can be staple non-woven, melt-blown, spunlaid, flashspun or any other suitable non-woven material. In some embodiments, the non-woven is suitable for use in a face mask. Typically, suitable non-woven face masks are manufactured from polypropylene which is considered to have low lung toxicity. Typically, the polypropylene fibres are not chemically bonded since chemical bonding agents may de-gas and be breathed in, for example.

Where the substrate is for use not as a face mask, the non-woven may be any type of non-woven, including chemically bonded, and not limited to any specific polymer.

The non-woven fabric may be manufactured by any of the current and well established methods including, but not limited to melt-blown, spun-bond, needle-punched, thermobonded, chemical bonded or any other suitable method.

Additionally, it may be desirable to combine non-woven fabrics of different polymers and/or fibre length, diameter and void space size and areal weight to create a single fabric with different properties such as, for example, void space, through its cross section.

In one embodiment the antimicrobial air-permeable substrate is a fibrous material such as a non-woven material.

The substrate may comprise or consist of polypropylene (PP) fibres, polyethylene, polyethylene terephthalate (PET), polytetrafluoroethylene (PFTE), polyvinylidene fluoride (PVDF), polylactic acid (PLA), polyurethane (PU), polystyrene, polyamide, polycarbonate, cellulose, rayon, nylon and polyester fibres or a combination thereof.

Suitable substrates include hydrophilic and hydrophobic substrates as well as amphiphilic substrates and both synthetic and natural fibres, including but not limited to cotton, silk and bamboo.

In one embodiment the non-woven material consists of polypropylene.

In one embodiment the non-woven material consists of nylon.

Advantageously, polypropylene and nylon have a triboelectric effect which can be generated by motion, for example, when breathing through the substrate. This, along with other methods such as hyperosmosis, ion discharge, oxidative stress, nanoparticle penetration, pH change and nucleic acid binding (for example by polyphenols) can provide a mechanism through which the microorganism can be deactivated.

In some embodiments the fibres are recycled.

In some embodiment the fibres are recyclable.

Advantageously, the substrate of the present invention could be recycled because any pathogens that have come into contact with the substrate would be denatured. This is in direct contrast to the current situation where, for example, PPE is incinerated due to contamination.

Advantageously, the use of recycled and recyclable materials for single use materials (e.g. PPE garments) is highly desirable from an ecological perspective.

In some embodiments the substrate comprises polypropylene fibres that have been carded and/or thermo-bonded to create a nonwoven fabric.

As employed herein, gsm is a measure of the density of the substrate and refers to the SI unit grams per square metre (g/m²). Typically, the substrate has a density in the range 5 to 500 gsm or 5 to 300 gsm, such as approximately 10, 15, 20, 30, 40, 50, 60, 70, 80, 90, 100, 110, 120, 130, 140, 150, 160, 170, 180, 190, 200, 210, 220, 230, 240, 250, 260, 27, 280 or 290 gsm. For example, the substrate has a density in the range approximately 10 to 50 gsm, such as approximately 20 to 25 gsm.

The fabric may be any type of fibrous structure but is preferably a non-woven fabric of areal weight of between 5 to 10 grams per square meter (gsm) and 200 to 300 grams per square meter (gsm).

Areal weight as employed herein refers to a term typically used to describe composite materials.

Essentially, it is a measure of the weight of fibre per unit area of fabric. In the non-woven industry it denotes the mass per unit area of a single ply of dry reinforcement fabric. In general, the density of the material is expressed as gsm, however, in some contexts areal weight may be used to describe non-woven material.

Non-woven fabric and non-woven material are used interchangeably herein.

Dry powder as employed herein refers to a particulate ingredient that is impregnated into the substrate by any suitable means such that it penetrates the substrate. It is referred to as dry powder because it is not introduced by solvating and soaking the substrate.

In one embodiment the dry powder is not introduced into the substrate by wetting the substrate with a solution in which the dry powder is dissolved.

As employed herein maximum particle size refers to an average of the maximum particle size of a dry powder. Where the particle are not uniform in shape, this is measured across the largest dimension. The particle size is taken as an individual particle size. Where agglomerations occur, the individual particles in the agglomeration are considered, not the agglomeration as a whole.

Typically, the dry powder is particulate and does not agglomerates when stored in dry conditions.

Generally, the particles are uniform in size.

Typically, the maximum particle size is not more than 500 μm (micrometres, microns). Such as not more than approximately 450, 400, 350, 300, 250, 200 μm. For example, not more than approximately 190, 180, 170, 160, 150, 140, 130, 120, 110, 100, 90, 80, 70, 60, 50, 40, 30, 20 or 10 μm. For example, not more than 150 μm. Such as not more than 110 μm.

In one embodiment the maximum particle size is not more than 110 μm.

In general, smaller particle sizes are desirable as they present a larger surface area. However, this is balanced against the possibility of being inhaled, which is to be avoided. Smaller particles may also disperse out of the substrate over time or in use. Particle encasement (as described below) can be employed to reduce this.

Preferably, the active compound or compounds are in powder form and within an average particle size range of between 1 micrometre (1 μm) and 500 micrometres (500 μm) although larger average particle sizes may also be used or a combination of particle sizes, depending upon the application of the final functionalised fabric. In some embodiments, the particles may be nanoparticles.

In some embodiments the particles may be crystals.

The present inventors were surprisingly able to produce a substrate that contains a high concentration of dry powder particles impregnated within it. Historically, it has been difficult to obtain high concentrations of dry powder impregnations into substrates and wet soaking (wet methods) of the substrate with a solution or suspension of the particles which is subsequently dried have failed to get meaningful concentrations of particles into the substrates.

As disclosed herein, the present inventors have been able to impregnate previously unobtainable levels of dry powder into the substrate to provide a novel substrate comprising at least 20% w/w dry powder.

Typically, the substrate comprises at least 20% w/w dry powder, such as approximately 25, 30, 35, 40, 45, 50, 55, 60, 65, 70, 75, 80 or 85% w/w dry powder. For example, approximately 40 to 80% w/w dry powder or 50 to 70% w/w dry powder.

In one embodiment the substrate comprises at least 30% w/w dry powder.

In one embodiment the substrate comprises at least 40% w/w dry powder.

In one embodiment the substrate comprises at least 50% w/w dry powder.

In one embodiment the substrate comprises at least 60% w/w dry powder.

In one embodiment the substrate comprises at least 70% w/w dry powder.

In one embodiment the substrate comprises up to 80% w/w dry powder.

In one embodiment the substrate comprises up to 75% w/w dry powder.

In one embodiment the substrate comprises up to 70% w/w dry powder.

In one embodiment the substrate comprises up to 65% w/w dry powder.

In one embodiment the substrate comprises up to 60% w/w dry powder.

In one embodiment the substrate comprises up to 55% w/w dry powder.

In one embodiment the substrate comprises up to 50% w/w dry powder.

Advantageously, the more dry powder (active ingredient) that can be impregnated into the substrate, the more effective it will be.

Expressed as areal weight, the actual areal weight of active compound or compounds impregnated within the fabric may range between 1% wt to 300% wt.

For clarity, and by way of example, if the active compound or compounds are impregnated into a non-woven fabric of say, 60 gsm at an average weight of 30 gsm then the active compound or compounds can be said to be impregnated at 50% wt.

Where the substrate is a non-woven material, to functionalise the fabric with the active compound or compounds, the preferred method is described in WO2016108039A1 (which is incorporated in its entirety herein by reference) such that the active compound or compounds are impregnated into the fibrous structure of the fabric so that the active compounds reside in the void spaces between the fibres of the fabric. It is considered that this method may have additional application where the substrate is not a non-woven material.

The dry powder can be considered to be an active ingredient in that it is not inert and plays a role in imparting, or increasing, the antimicrobial (particularly antiviral) properties of the substrate.

The active compound or active ingredient may comprise (or consist of), but is not limited to, glucose, carbon allotropes, acidic powders such as citric acid, salts including organic and inorganic salts such as sodium chloride, sodium bicarbonate, potassium sulphate, potassium chloride, or ammonium sulphate, quaternary ammonium compounds, magnesium stearate, activated carbon, silicon dioxide, copper, silver, zinc oxide, aluminium oxide, titanium dioxide, zeolites and surfactants singly or in any combination or ratio.

In one embodiment the dry powder is a salt, such as NaCl.

Advantageously, sodium chloride is widely available and inexpensive. It is non-toxic and safe to use against human skin. It is also easily disposed of without damaging the environment.

In one embodiment the dry powder is a blend of two or more dry powders.

In one embodiment the blend is a blend of NaCl and NaHCO₃.

In one embodiment the ration of NaHCO₃ to NaCl does not exceed 1:9. That is, 1 part NaHCO₃ to 9 parts NaCl, or a blend of 90% NaCl to 10% NaHCO₃.

Suitable uses for the antimicrobial substrate disclosed herein include using it as a functionalised layer in a multi-layer material. In general, the substrate may be joined to or collocated next to at least one layer of a substrate that does not comprise the dry powder.

For example, where the substrate is a non-woven material impregnated with dry powder to make a functionalised layer, that functionalised layer may be sandwiched between two layers of non-woven that is not impregnated with the dry powder. This sandwiched material may comprise 3 or more ply, where the outermost plies are each independently non-functionalised material.

In one embodiment the substrate is sandwiched next to at least one layer of non-functionalised substrate.

Non-functionalised as employed herein refers to a substrate that does not comprise the dry powder.

In some embodiments multiple layers of the substrate as disclosed herein are used to create a multi-layer material.

As described above, it is desirable to prevent the dry powder from redistributing once it is impregnated into the substrate. One way to achieve this may be through the use of fine, particle-filtering barrier layers on the outsides of the substrate, which may be less air-permeable.

Another option is to use particle encasement to assist with retaining the dry powder in place. The encasement may be achieved using any suitable means including, but not limited to, small, tessellating welded cages or hot calendaring of the substrate.

In some embodiments the particle encasement shrinks the fibres in a non-woven material and more tightly binds the particles within the void spaces.

In some embodiments the particle encasement also imparts a degree of rigidity to the non-woven material. It is important to balance drapability of the substrate with particle encasement and the degree to which this is balanced may depend on the intended use of the substrate.

Alternatively, or additionally, compartmentalisation can be used to assist with retaining the dry powder in place. The compartmentalisation may be achieved using any suitable means including, but not limited to, stitching, melting, compressing, welding or hot calendaring of a pattern onto the multi-layer material (for example, as shown in FIG. 3).

The compartmentalisation pattern may take any form including, but not limited to, dots, squares, rectangles, triangles, hexagons.

In one embodiment the substrate is compartmentalised using hexagons.

Suitable methods of impregnating a substrate with a dry powder are disclosed in WO2016/108039. Typically, such methods involve first dispersing the dry powder onto the surface of the substrate, such as a non-woven material and then applying some form of energy to the substrate to permit the dry powder to penetrate the void spaces of the substrate.

Suitable methods of dispersing the dry powder include, but are not limited to: dispersing the dry powder onto the surface of air-permeable substrate by a controllable mechanical means such as precision scatter coating, whereby the particles are mechanically distributed onto the surface via a rotary screen. Other types of scatter coating mechanisms are also suitable. Alternatively, powder spraying, vibrating particle feeder systems (for example, with electromagnetic or vibrator motor drives) or electromagnetic drive feeders may be used.

Using one of aforementioned methods or any other method of controlled dispersion, the particles may be dispersed across the entire surface of the air permeable substrate, or only across selected, pre-determined areas of the air permeable substrate depending upon the design requirements of the end manufactured product.

A number of methods are suitable for impregnating the particles into the air permeable substrate. These include but, are not limited to, externally applied vibration energy (VE), alternating electrical field (AEF), high frequency vibration via, for example, an ultrasonic vibrating sonotrode or vacuum applied to the opposite side of the substrate to the dry powder to draw particles into the substrate, or a combination of the foregoing.

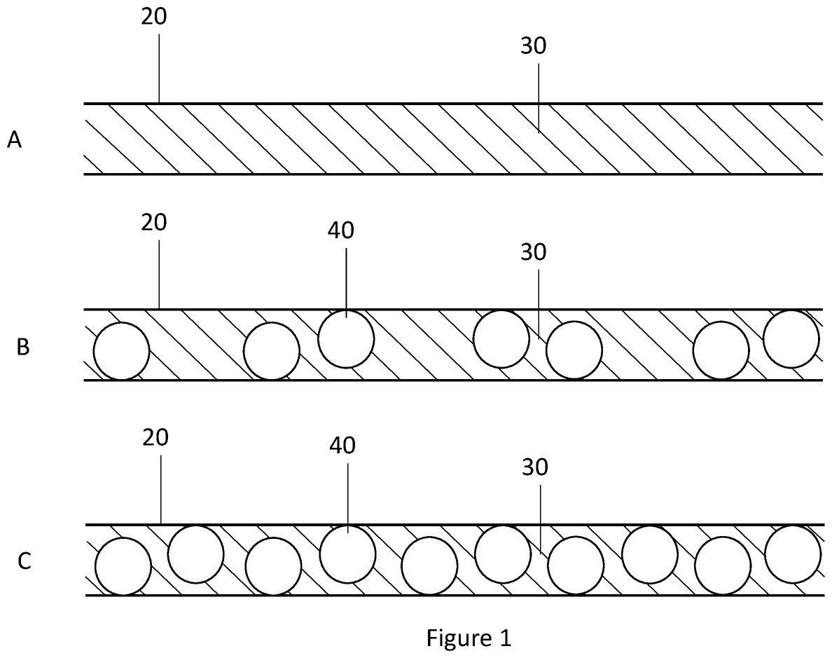

Referring first to FIG. 1, there is shown a schematic cross section through an air-permeable substrate generally indicated 20. The substrate is indicated as a single layer, although multiple layers are considered within the scope of the disclosure. In FIG. 1A the substrate is shown as a fibrous substrate with fibres indicate 30. FIG. 1B indicates a single layer of substrate 20 in which a medium concentration of dry powder 40 is impregnated. FIG. 1C indicates a single layer of substrate 20 in which a high concentration of dry powder 40 is impregnated.

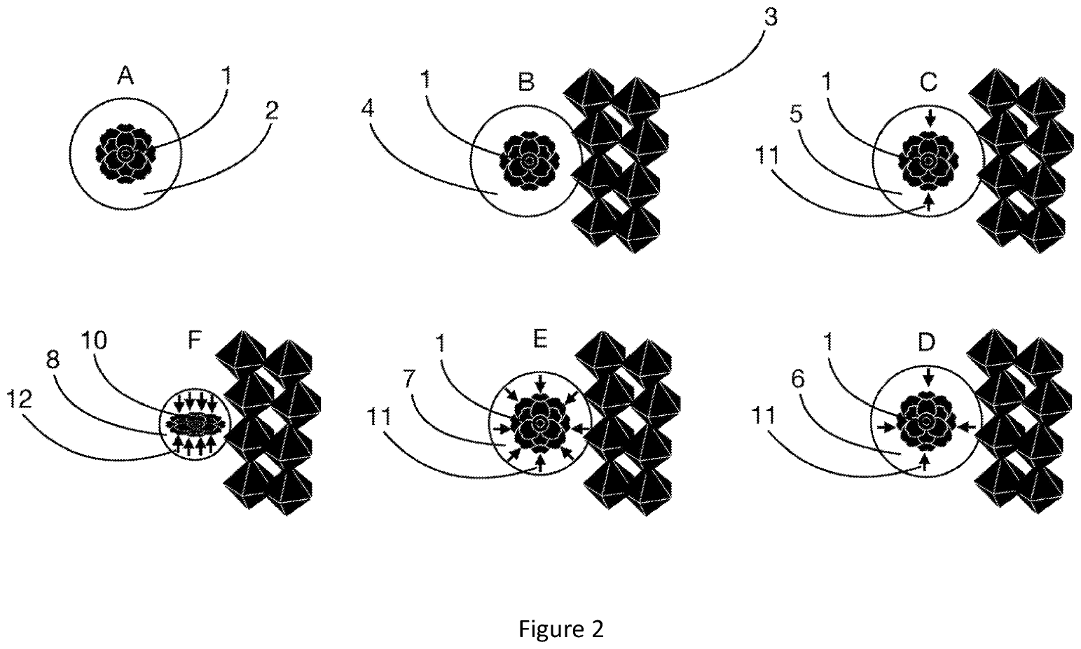

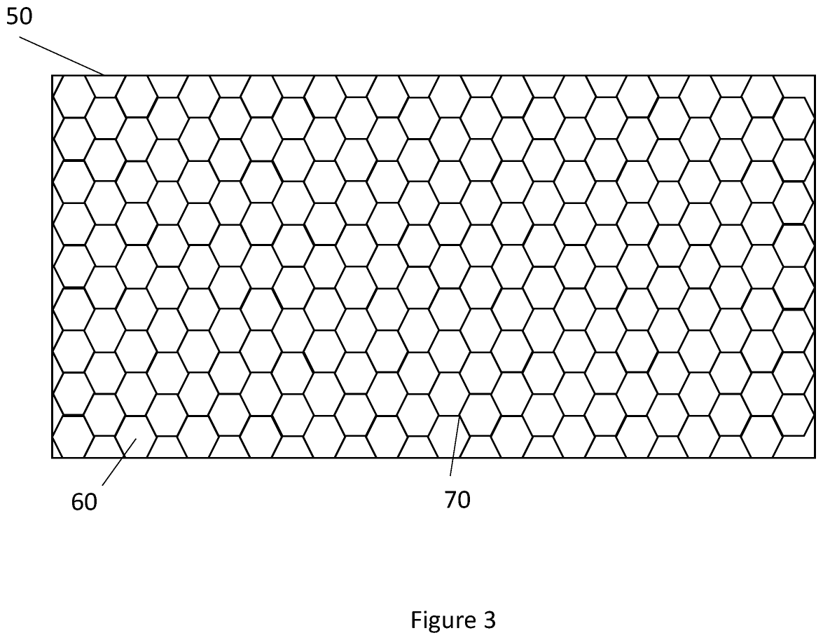

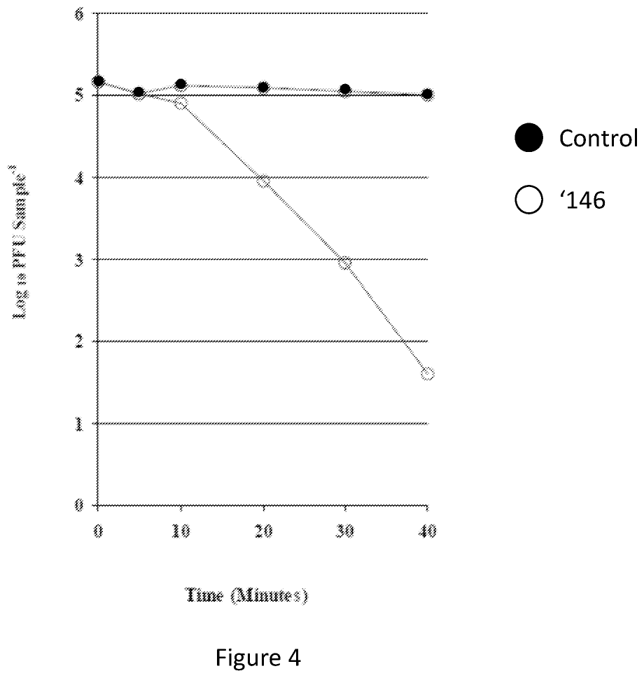

Referring now to FIG. 2 there is indicated a schematic representation of a mechanism of deactivating a microorganism by the antimicrobial substrate according to the disclosure.

Without wishing to be bound by theory, it is believed that the mechanism for deactivating virus and other pathogens within the functionalised fabric is as follows, described with reference to FIG. 2.

Human airborne virus is mostly transmitted via human mucus when an infected person coughs, sneezes or otherwise expels air from their respiratory system. Human mucus contains>96% liquid water.

Referring to FIG. 2, there is shown in section A, a representation of an airborne virus 1 within human mucus 2 which could be in the form of a droplet or aerosol.

Section B of FIG. 2 is a representation of the airborne virus 1 contacting the activated powder (dry powder) 3 located within the substrate, such as a non-woven fabric (not shown for clarity) such that the water content of the human mucus immediately begins to dissolve the active dry powder 3 upon contact to become a low salt saline hypertonic solution 4.

Section C of drawing 1 is a representation of the continued dissolution of the active powder 3 thereby further increasing the salinity of the water, decreasing the isotonic solution content of the human mucus 5 and consequently increasing the osmotic pressure 11 on the virus 1 contained within.

Section D of drawing 1 is a further representation of the continued dissolution of the active powder 3 further increasing the salinity of the water content, decreasing the isotonic solution of the human mucus 6 and thereby yet further increasing the osmotic pressure 11 on the virus 1 contained within.

Section E of drawing 1 is a further representation of the continued dissolution of the active powder 3 further increasing the salinity of the water content, decreasing the isotonic solution to the solubility limit of the water content of the human mucus 7 by the active powder 3 and thereby further significantly increasing the osmotic pressure 11 to the point of hyper-osmosis on the virus 1 contained within whilst simultaneously, the water content of human mucus rapidly begins to evaporate into the surrounding atmosphere as the active compound 3 begins to recrystallise.

Section G of drawing 1 shows point where the hyperosmotic pressure within the human mucus and the pressure of recrystallisation 12 of the active powder 3 has ruptured the viral envelope of the now deactivated virus.

Advantageously, where the dry powder is a salt, it acts as a desiccant.

Referring now to FIG. 3 there is indicated an embodiment according to the present disclosure wherein the substrate 50 (shown face on) has been compartmentalised. In this embodiment the compartments 60 are hexagonal in shape. The “walls” 70 of the compartments inhibit transfer of dry powder within the substrate.

Referring now to FIG. 4 there is shown a graph indicating the deactivation of viral particles by the impregnated substrate relative to an unimpregnated control. In the specific embodiment, 46 g of NaCl where impregnated into 100 g of substrate to give a 31.5% w/w antimicrobial substrate. It can be seen that the number of PFU (virus) declines significantly within 40 minutes of being in contact with the impregnated (activated) substrate.

In the context of this specification “comprising” is to be interpreted as “including”.

Approximately as employed herein is defined as ±10%.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

### EXAMPLES

### Introduction

Testing antimicrobial agents for efficacy against viruses is usually performed using surrogates of the main target species (often those that target mammals). Although the viruses are relatively robust, the host cells used to detect and quantify them are not (cells grown in culture are mainly used for this purpose rather than whole target species). Due to the large and relatively irregular form of the cells in the lawns used for this purpose, the assay techniques lack the relative precision associated with the techniques used to enumerate bacteria. However, there are a number of bacteriophage species that are also structural analogues of many different mammalian, avian, piscine and plant viruses and that are used as surrogates in testing. This includes species such as phi6 (which infects certain species of the bacterial genus, Pseudomonas) that is structurally very similar to the mammalian virus Coronavirus and which exhibits very similar characteristics with respect to environmental persistence and sensitivity to biocides as them.

Tests based on bacteriophages can be performed with relative ease (compared with using mammalian viruses) and significantly lower associated rates of failure of the test model (and hazards to the operator when viruses that are pathogenic to man are used) than many other viruses (the host cell lines of which are highly susceptible to contamination and loss of viability). The technique also employs techniques that are similar in precision and robustness to those associated with many bacterial tests (due to the similarity of the methods employed). Tests on biocides, and treated articles using bacteriophages can be highly indicative of the outcome to be expected for other viruses with a similar structure.

This example summarises a proof of principle study to assess the antiviral efficacy of fabric formulations against phi6 bacteriophage (enveloped bacterial virus) in the presence of a low level soiling medium using a method based on ISO 18184:2019.

Test Materials

Samples of component fabric (30 gsm polypropylene, impregnated ultrasonically and compartmentalised) either unfortified or fortified with an antiviral additive were tested alongside sample of unfortified polystyrene to act as a reference material. All samples were held in the dark at 20° C. prior to testing.

Method

A proof of principle study into the basic determination of antiviral efficacy against an enveloped (phi6) bacteriophage was determined was determined using a method based on ISO 18184:2019.

3.1 Preparation of Test Inoculum

Individual suspensions of the phage listed in Table 1 were prepared. The host bacterial strains were held as primary stock cultures at 5° C.±3° C. prior to use. The host organism was sub-cultured in 50 mL Tryptone Soy Broth (TSB) and incubated under constant agitation on an orbital shaker with a 40 mm throw at 200 rpm at 28° C.±2° C. for approximately 5 hours. An aliquot (5 mL) of a bacteriophage stock suspension was then added to the resultant culture and incubated under constant agitation for a further 3 hours at 28° C.±2° C.

The resultant virus infected culture was separated into a supernatant and pelleted cells/cell debris by centrifugation (1800 g for 15 minutes at ca 21° C.). The supernatant was then filtered through a 0.45 μm sterile membrane filter to remove any residual bacteria and cell debris.

The titre of bacteriophage in the filtrate was determined using dilution plaque count by transferring 1 mL of the appropriate dilution into an aliquot (5 mL) molten (48° C.) Tryptone Soy Agar (TSA) seeded with the bacterial host strain (ca 10⁷CFU mL⁻¹) which was then overlaid on pre-poured plates of TSA. The filtrate was then stored at 5° C.±3° C. The overlay plates were then incubated for 48 hours at 28° C.±2° C. and the number of plaques present were counted. These counts were used to determine the titre of bacteriophage in the stored filtrates.

Immediately prior to use the filtrate was diluted to the concentration required using 0.3 g L⁻¹ Bovine Serum Albumin (BSA). The number of plaque forming units (PFU) in the resulting suspension was confirmed by dilution plate count as described above.

TABLE 1

| Phage Species | Strain Reference | Host | Strain Reference |
|---|---|---|---|
| phi6 (enveloped) | ATCC 21781 - | Pseudomonas | ATCC 21781 |
| | B1 | syringae | |

3.2 Test Method

An individual aliquot (20 μL) of the phage suspension as described above was held in intimate contact with a single replicate of the test fabrics supplied for 1 hour at 20° C.±2° C. and 55% relative humidity.

The size of the surviving population was determined using dilution plate count as described in Section 3.1. The test plates were incubated at 28° C. for 48 hours, and then plaque forming units were counted.

An additional replicate unfortified textile were also inoculated in the manner described above but were then analysed immediately for the size of microbial population present to provide 0-time control data.

All data were converted to plaque forming units (PFU) sample⁻¹ and then transformed to provide a data-set that conformed to a Gaussian distribution.

Results/Discussion

The results as PFU sample⁻¹ are shown in Table 2 and FIG. 4.

TABLE 2
Activity Against phi6 (Enveloped Bacteriophage) (Recovery of 1 Replicate as Plaque Forming Units Sample⁻¹)

| | | Contact Time (Minutes) | | | | | | Reduction from Initial (%) | | | | |
|---|---|---|---|---|---|---|---|---|---|---|---|---|
| Sample | Soiling | 0 | 5 | 10 | 20 | 30 | 40 | 5 | 10 | 20 | 30 | 40 |
| Polystyrene | 0.3 g L⁻¹ | 1.5 × 10⁵ | 1.1 × 10⁵ | 1.3 × 10⁵ | 1.3 × 10⁵ | 1.1 × 10⁵ | 1.0 × 10⁵ | 27.59 | 10.34 | 13.79 | 24.14 | 31.03 |
| ′146 | BSA | — | 1.1 × 10⁵ | 8.0 × 10⁴ | 9.1 × 10³ | 9.0 × 10² | 4.0 × 10¹ | 27.59 | 44.83 | 93.72 | 99.38 | 99.97 |

*Theoretical limit of detection is 5 PFU sample⁻¹

It can be seen from the results in Table 2 above that the number of virions of phi6 suspended in 0.3 g L⁻¹ BSA held in contact with the polystyrene reference material and fabric declined by 0.2 orders of magnitude over the 40 minute contact period compared to the initial population

The number of virions of phi6 suspended in 0.3 g L⁻¹ BSA held in contact with the samples of ′146 declined by 0.1, 0.3, 1.2, 2.2 and 3.6 orders of magnitude after 5 minutes, 10 minutes, 20 minutes, 30 minutes and 40 minutes respectively compared to the initial population.

What is claimed is:

1. An antimicrobial air-permeable substrate having density in a range of 5 grams per square meter (gsm) to 500 gsm comprising a dry powder with a maximum particle size of 500 micrometers (μm) in the amount of at least 20 percent by weight (w/w).

2. The antimicrobial air-permeable substrate according to claim 1 wherein the substrate is a non-woven fibrous material.

3. The antimicrobial air-permeable substrate according to claim 1 wherein the substrate comprises polypropylene fibres.

4. The antimicrobial air-permeable substrate according to claim 1 wherein the substrate comprises polypropylene fibres that have been carded and/or thermo-bonded to create a non-woven fabric.

5. The antimicrobial air-permeable substrate according to claim 1 wherein the dry powder is a salt.

6. The antimicrobial air-permeable substrate according to claim 1 wherein the dry powder is a blend of two or more dry powders.

7. The antimicrobial air-permeable substrate according to claim 6 wherein the blend of two or more dry powders is a blend of sodium chloride (NaCl) and sodium bicarbontate (NaHCO₃).

8. The antimicrobial air-permeable substrate according to claim 7 wherein a ratio of NaHCO₃ to NaCl is at most 1:9.

9. The antimicrobial air-permeable substrate according to claim 1 wherein the substrate is joined to or collocated next to at least one layer of a substrate that does not have the dry powder.

10. The antimicrobial air-permeable substrate according to claim 1 wherein the substrate is a non-woven material with a density in the range 15 to 30 grams per square meter (gsm), a maximum particle size is at most 110 micrometers (μm) and the dry powder is present in the amount of at least 30 percent by weight (w/w).

11. The antimicrobial air-permeable substrate according to claim 1 wherein the substrate has undergone particle encasement.

12. The antimicrobial air-permeable substrate according to claim 1 wherein the substrate comprises compartments.

13. The antimicrobial air-permeable substrate according to claim 12 wherein the compartments are hexagonal.

14. A multi-layered material comprising at least one layer of a permeable substrate having density in a range of 5 grams per square meter (gsm) to 500 gsm comprising a dry powder with a maximum particle size of 500 micrometers (μm) in the amount of at least 20 percent by weight (w/w).

15. Use of the antimicrobial air-permeable substrate according to claim 1 to kill, denature or otherwise deactivate a microorganism.
